(19) United States
(12) Patent Application Publication
(10) Pub. No.: US 2016/0120488 A1
(43) Pub. Date: May 5, 2016

(54) Intraoral X-Ray Detector Assembly And Intraoral X-Ray Imaging Apparatus Using Same

(71) Applicant: RE-MEDI CO., LTD, Chuncheon-si, Gangwon-do (KR)

(72) Inventors: Rena LEE, Seoul (KR); Jaehee WOO, Gyeonggi-do (KR)

(21) Appl. No.: 14/441,769

(22) PCT Filed: Nov. 8, 2013
(86) PCT No.: PCT/KR2013/010138

§ 371 Date: Jun. 26, 2015

(30) Foreign Application Priority Data
Nov. 8, 2012 (KR) 10-2012-0125954

(51) Int. Cl. A61B 6/14; A61B 6/00

(52) U.S. Cl. CPC A61B 6/145; A61B 6/4411; A61B 6/4233; A61B 6/4057

(57) ABSTRACT

Provided is an intraoral X-ray detector assembly that provides X-ray detection signals to an X-ray imaging apparatus main body, the assembly may comprises: an X-ray irradiation means disposed to emit X-rays toward a dentition to be imaged within an oral cavity of a patient; a close-contact means that can bring the X-ray detection means capable of detecting the X-rays close to the patient's face and can fix the X-ray detection means so as to face an X-ray emission direction of the X-ray irradiation means; and a support means that supports a first shaft extending to the X-ray irradiation means and a second shaft extending to the close-contact means and is mechanically coupled to a connection means between the X-ray imaging apparatus main body.

### CROSS-REFERENCE TO RELATED APPLICATION

This Application is a Section 371 National Stage Application of International Application No. PCT/KR2013/010138, filed Nov. 8, 2012, the contents of which are hereby incorporated by reference in its entirety.

### BACKGROUND OF THE INVENTION

1. Technical Field

The present invention relates to an X-ray imaging apparatus. More particularly, it relates to an X-ray detector assembly for use in intraoral X-ray imaging apparatus.

2. Background Art

An X-ray imaging apparatus is as apparatus that irradiates toward X-ray to an object, and detects the image projected from the object to check an internal state of the object. The X-ray imaging apparatus has also been utilized in applications for imaging structures of dentition and oral cavity in dentistry. It is possible to check the decay and gum status from the X-ray image pickup image.

Existing intraoral X-ray imaging apparatuses are types in which a detector is placed inside the oral cavity, and when an X-ray generator emits X-rays while moving to draw a semicircle around a chin outside the oral cavity, the detector inside the oral cavity detects X-rays that have passed through the teeth.

In such types, since the detector is fixed inside the oral cavity, there is a limit in the size of the detector, it causes a lot of inconvenience to a patient, and since an X-ray source is directed toward the interior of the human body, unnecessary X-ray exposure occurs.

Recently, an intraoral X-ray imaging apparatus which has remarkably improved these problems has been developed. As its basic principle, a subminiature X-ray generator is inserted into the oral cavity to detect the X-rays from the outside.

Such a type has an advantage in which, since the X-ray device is small enough to be inserted into the oral cavity, patient's discomfort is reduced, and by directing the X-ray source toward the outside of the human body, it is possible to minimize the X-ray exposure. However, in order to obtain a high-quality of image, there is a need for development of both a scanning method and a detector depending on various intraoral structure and size.

Thus, there is still a need for a detection system that can enhance the quality of images and can take images within a short time, while maintaining the advantages of the intraoral X-ray imaging apparatus capable of minimizing the discomfort of the patient and X-ray exposure.

### DISCLOSURE

### Technical Problem

An aspect of the present invention provides an intraoral X-ray detector assembly that can minimize the X-ray exposure and discomfort of patient, while being able to cope with various structures and sizes of oral cavity, and can rapidly take X-ray images.

### Technical Solution

According to an aspect of the present invention, there is provided an intraoral X-ray detector assembly that provides X-ray detection signals to an X-ray imaging apparatus main body, the assembly including: an X-ray irradiation means disposed to emit X-rays toward a dentition to be imaged in an oral cavity of a patient; a close-contact means that can bring the X-ray detection means capable of detecting the X-rays close to the patient's face, and can fix the X-ray detection means so as to face an X-ray emission direction of the X-ray irradiation means; and a support means that supports a first shaft extending to the X-ray irradiation means and a second shaft extending to the close-contact means and is mechanically coupled to a connection means between the X-ray imaging apparatus main body.

According to an embodiment, the close-contact means may include an intermediate plate coupled to the second shaft; a detection means support plate to which the X-ray detection means is coupled; and an elastic body interposed between the intermediate plate and the detection means support plate.

According to on an embodiment, the elastic body is a spring, the close-contact means may further include a guide that guides a compression or pulling direction of the spring between the intermediate plate and the detection means support plate.

According to an embodiment, the support means may be mechanically coupled to the connection means so as to be attachable and detachable.

According to an embodiment, the support means may be mechanically coupled to the connection means so as to be attachable and detachable, when the X-ray irradiation means is in a direction positioned to face the patient's dentition.

According to an embodiment, the support means may be mechanically coupled to the connection means so as to be rotatable about a first rotary axis that coincides with an extension direction of the first shaft.

According to an embodiment, the second shaft may be mechanically coupled to the support means so as to be rotatable about the second rotary axis intersecting with the first rotary axis

According to an embodiment, the second shaft may be mechanically coupled to the support means so as to be rotatable about the second rotary axis, between a position where the close-contact means faces the X-ray irradiation means and a movement position for changing the image pickup position of the close-contact means.

According to an embodiment, the X-ray detection means may be one of a one-dimensional X-ray line detector or a two-dimensional X-ray pixel array.

### Advantageous Effect

According to an intraoral X-ray detector assembly of the present invention, since a bulky detector is located outside an oral cavity of a patient and a small X-ray collimator is inserted into the oral cavity of the patient, discomfort of the patient can be reduced.

According to the intraoral X-ray detector assembly of the present invention, since the left and right sides of the oral cavity are separately imaged, the X-ray collimator is configured to emit X-rays only in the direction in which the X-ray detector is located, which makes it possible to reduce the overall X-ray exposure dose.

According to the intraoral X-ray detector assembly of the present invention, since the X-ray detector is positioned in close contact with or close proximity to the patient's skin, it is possible to further reduce the intensity of X-ray necessary for imaging.

According to the intraoral X-ray detector assembly of the present invention, since it is possible to adjust the relative position of the X-ray collimator with respect to the X-ray detector, it is possible to cope with the oral cavity structure and size of the various patients.

According to the intraoral X-ray detector assembly of the present invention, after the completion of imaging of any one side of the left and right sides of the oral cavity, it is possible to apply the X-ray detector to the imaging of the opposite side, by immediately rotating the X-ray detector.

### BRIEF DESCRIPTION OF THE DRAWINGS

The above objects, other features and advantages of the present invention will become more apparent by describing the preferred embodiments thereof with reference to the accompanying drawings, in which:

FIGS. 1 and 2 are diagrams illustrating an intraoral X-ray detector assembly according to an embodiment of the present invention.

FIG. 3 is a diagram illustrating a case where an X-ray detection means of the intraoral X-ray detector assembly according to an embodiment of the present invention is brought close-contact with the X-ray irradiation means which is inserted into the oral cavity.

### PREFERRED EMBODIMENTS OF THE INVENTION

According to an aspect of the present invention, there is provided an intraoral X-ray detector assembly that provides X-ray detection signals to an X-ray imaging apparatus main body, the assembly including: an X-ray irradiation means disposed to emit X-rays toward a dentition to be imaged in an oral cavity of a patient; a close-contact means that can bring the X-ray detection means capable of detecting the X-rays close to the patient's face, and can fix the X-ray detection means so as to face an X-ray emission direction of the X-ray irradiation means; and a support means that supports a first shaft extending to the X-ray irradiation means and a second shaft extending to the close-contact means and is mechanically coupled to a connection means between the X-ray imaging apparatus main body.

In regard to embodiments of the invention disclosed in the text, the particular structural or functional descriptions are merely exemplified for the purposes of illustrating an embodiment of the present invention, and embodiments of the present invention can be embodied in various forms and should not be construed as being limited to the embodiments described herein.

Hereinafter, preferred embodiments of the invention will be described in more detail with reference to the accompanying drawings. The same components in the drawings are denoted by using the same reference numerals, and the repeated explanation of the same components will be omitted.

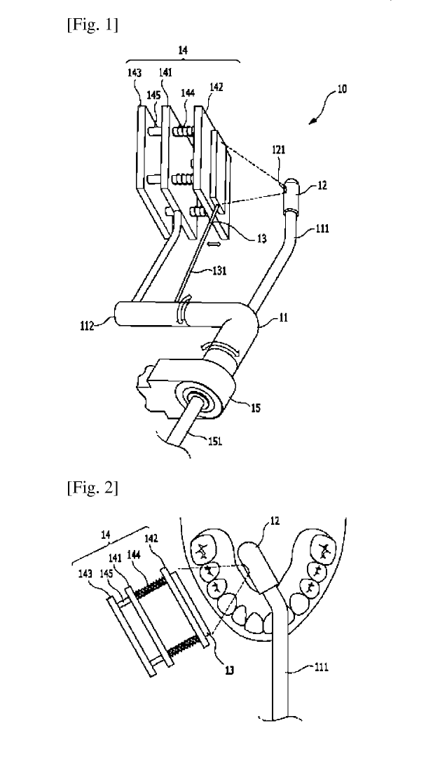

FIGS. 1 and 2 are diagrams illustrating an intraoral X-ray detector assembly according to an embodiment of the present invention.

FIGS. 1 and 2 are diagrams illustrating a case where an intraoral X-ray detector assembly 10 is located so as to be able to image a left dentition and a right dentition of the patient.

Referring to FIGS. 1 and 2 together, the intraoral X-ray detector assembly 10 can include a support means 11, an X-ray irradiation means 12, an X-ray detection means 13, a close-contact means 14 and a connection means 15 so as to be able to minimize the discomfort of the patient, while minimizing the X-ray exposure dose, by generating X-rays in the oral cavity toward the X-ray detection means located near the cheek outside the oral cavity.

In other words, the intraoral X-ray detector assembly 10 is intended to provide an X-ray detection signal to a main body (not shown) of the X-ray imaging apparatus, and can include an X-ray irradiation means 12 disposed to emit X-rays toward a dentition to be imaged in an oral cavity of a patient; a close-contact means 14 that can bring the X-ray detection means 13 capable of detecting the X-rays close to the patient's face, and can fix the X-ray detection means so as to face an X-ray emission direction of the X-ray irradiation means 12; and a support means 11 that supports a first shaft 111 extending to the X-ray irradiation means 12 and a second shaft 112 extending to the close-contact means 14 and is mechanically coupled to a connection means between an X-ray imaging apparatus main body (not shown).

Specifically, the connection means 15 is a portion connected to a body of an intraoral imaging apparatus (not shown) that includes various components for supplying electric power to the intraoral X-ray detector assembly 10 and receiving and processing the X-ray detection data, and as an example, a second cable 151 can include a power that supplies electric power to the X-ray irradiation means 12 or other electrically driven elements, and a data cable that transmits the X-ray detection signal detected by the X-ray detection means 13 to an intraoral X-ray imaging apparatus (not shown) therein.

According to the embodiment, the support means 11 can be mechanically coupled to the connection means 15 so as to be rotatable with a predetermined first rotational angle on the basis of a first rotational axis which at least substantially coincides with an extension direction of a first shaft 111, and so as to be able to keep a rotated position after the rotation. At this time, when the support means 11 rotates, a second shaft 112 extending from the support means 11 can also rotate together.

For example, the support means 11 is shown in FIG. 1 in a fixed state so that the close-contact means 14 is fixed so as to be located on a left side (on the basis of a direction of seeing FIG. 1) of the X-ray irradiation means 12. After rotating by a predetermined angle on the basis of the first rotary axis, the close-contact means 14 is in a fixed state so as to be located on the right side of the X-ray irradiation means 12, and at this time, an X-ray emission direction of the X-ray irradiation means 12 also rotates towards the other dentition of the patient along with the rotation of the first shaft 111.

Thus, in one embodiment, the support means 11 can rotate by the first rotation angle on the basis of the first rotational axis, while maintaining the mechanical contact with the connection means 15.

In this case, depending on the embodiment, the support means 11 can restore a mechanical engagement state with the connection means 15 at any time at a position of being rotated at any angle within the first rotation angle.

Rotation of the support means 11 by the first rotation angle may be manually embodied, that is, embodied in a way of turning the support means on the basis of the first rotational axis by hand, and may be embodied in an automated way using an electric motor or a hydraulic or pneumatic rotor.

In another embodiment, first, after the engaged state of the support means 11 with the connection means 15 is released in a direction in which the X-ray irradiation means 12 is positioned to face one dentition of the patient, the support means 11 rotates by the first rotation angle on the basis of the first rotational axis. When the X-ray irradiation means 12 is positioned to face the other dentition this time, it can be engaged with the connection means 15 again. In other words, in this embodiment, after being disengaged from the connection means 15, the support means 11 can be engaged with the connection means 15 by turning the direction on the basis of the first rotational axis.

Meanwhile, for example, an I-shaped first shaft 111 extends from the support means 11 along the first rotational axis so as to be able to position the X-ray irradiation means 12 at a suitable position within the oral cavity. In other words, one end of the first shaft 111 is fixed to the support means 11, and the other end thereof is fixed to the X-ray irradiation means 12. At this time, the other end of the first shaft 111 may be bent so that the X-ray irradiation direction of the X-ray detection means 12 generally faces the X-ray irradiation means 13.

The interior of the first shaft 111 is preferably empty, and a power supply cable for the X-ray irradiation means 12 can pass through the empty interior.

The X-ray irradiation means 12 can be embodied as a collimator designed to induce the emission of the X-ray to a predetermined position and direction and an X-ray generating element mounted thereon, and it can be referred to as an X-ray collimator.

The X-ray irradiation means 12 can have a shape like a tube with grooves capable of being fixed by teeth and tongue of the patient so as not to move, for example, capable of being pressed by the teeth, when taking an image in the oral cavity.

The tube-shaped X-ray irradiation means 12 is fixed to the other end of the first shaft 111 at one end portion of the tube, and the other end portion of the tube can have an open X-ray emission port 121 that is opened to face the X-ray detection means 13.

The X-ray irradiation means 12, for example, can be manufactured so that the main irradiation direction of X-rays generally faces the X-ray detection means 13, by emitting the X-rays generated by hitting of electrons having strong energy emitted from a negative electrode against a target material to the outside after passing through an X-ray smoothing filter, when a positive electrode and a negative electrode are formed in a small vacuum glass tube, a specific target material is attached to the end of the positive electrode and a strong voltage is applied between the electrodes.

The X-ray irradiation means 12 can also be manufactured by using a metal casing or a ceramic insulator in place of the vacuum glass tube.

Further, a second shaft 112 can be fixed to the support means 11, for example, in an L-shape so that the close-contact means 14 supported by the support means 11 is spaced apart from the X-ray irradiation means 12 by a predetermined distance, while facing the X-ray irradiation means 12.

In other words, according to the embodiment, the second shaft 112, for example, can extend along the second rotary axis direction perpendicular to the first rotary axis and then can be bent in a direction parallel to the first rotary axis again, in the state of being formed integrally with the support means 11.

One side of the second shaft 112 is fixed to the support means 11, and the other side thereof is fixed to the close-contact means 14. At this time, the distal end of the second shaft 112 on the side facing the contact means 14 can be slightly bent outward, depending on the shape of the face.

In another embodiment, the second shaft 112 can be mechanically coupled to the support means 11 so as to be rotatable along the second rotary axis , for example, intersecting with, and for example, orthogonal to the first rotary axis, and furthermore so as to be able to maintain the rotational position after rotation. In other words, the second shaft 112 rotates with respect to the support means 11 on the basis of the second rotary axis, thereby being able to rotate the close-contact means 14, for example, so as to be located in a direction of protruding upward from a sheet plane of FIG. 1.

In the embodiments, a second shaft 112 mechanically coupled to the support means 11 in a rotatable manner can rotate on the basis of the second rotary axis, within a second rotation angle that may be defined between a position of causing the close-contact means 14 to face the X-ray irradiation means 12, and a position suitable to move to change the position of the close-contact means 14, while preventing the patient's face from touch before or after the X-ray image pickup.

The rotation of the second shaft 112 may be manually embodied, in other words, in a way of turning the second shaft 112 on the basis of the second rotary axis by hand, and the rotation may be embodied by an automated way by utilizing an electric motor or a hydraulic or pneumatic rotor.

For example, the close-contact means 14 is fixed to the other end of the second shaft 112. The close-contact means 14 is capable of bringing the X-ray detection means 13 embodied as a one-dimensional or two-dimensional X-ray detector into close-contact with the patient's dentition side by side.

For example, by utilizing a mechanical elastic force, when imaging the X-ray, the close-contact means 14 can ensure the detection position X-ray detection means 13 close to or close-contact with the patient's face as much as possible, and before or after the X-ray image pickup, the close-contact means 14 can space the X-ray detection means 13 away from the patient's face so the patient does not feel discomfort.

To this end, as an example, the close-contact means 14 can be embodied as an intermediate plate 141, detection means support plate 142, a stopper plate 143, an elastic body 144 and a guide 145.

The intermediate plate 141 is integrally fixed to the second shaft 112 of the close-contact means 14, and is formed with guide holes so that the guides 145 can pass through.

The X-ray detection means 13 is mounted on the detection means support plate 142. The X-ray detection means 13 can be fixedly attached to the other side surface of the two side surfaces of the detection means support plate 142, rather than a side surface adjacent to the patient's face.

Meanwhile, the detection unit support plate 142 can be bent in accordance with the shape of the patient's face on at least one side surface.

The elastic bodies 144 are interposed between the intermediate plate 141 and the detection means support plate 142, thereby being able to push out the detection means support plate 142 toward the X-ray irradiation means 12, on the basis of the intermediate plate 141, at least at the time of imaging.

The elastic bodies 144 can be embodied as a spring or the like, and the guides 145 can be interposed between the intermediate plate 141 and the detection means support plate 142 so as to guide the elastic direction of the elastic bodies 144.

At this time, when spacing the detection means support plate 142 away from the patient's face before or after the imaging, that is, when the elastic bodies 144 are compressed as much as possible, the distal ends of the guides 145 can protrude through guide holes (not shown) of the intermediate plate 141.

Conversely, in order for the guides 145 to limit the maximum expansion position of the elastic bodies 144 when bringing the detection means support plate 142 close to the patient's face during X-ray image pickup, a stop plate 143 can be included at the distal ends of the guides 145. Although the stopper plate 143, for example, is exemplified as a plate shape similar to the intermediate plate 141, a shape that prevents the distal ends of the guides 145 from passing through the guide holes of the intermediate plate 141, for example, even a flange-like shape is enough.

Next, the X-ray detection means 13 can be embodied as a film, a one-dimensional analog/digital X-ray line detector or a two-dimensional analog/digital X-ray pixel array detector. In a case where the X-ray detection means 13 is an analog/digital X-ray detector, the detection signal can be transmitted through an X-ray signal cable 131. The X-ray signal cable 131 can pass through an empty interior of the second shaft 112 according to the embodiment.

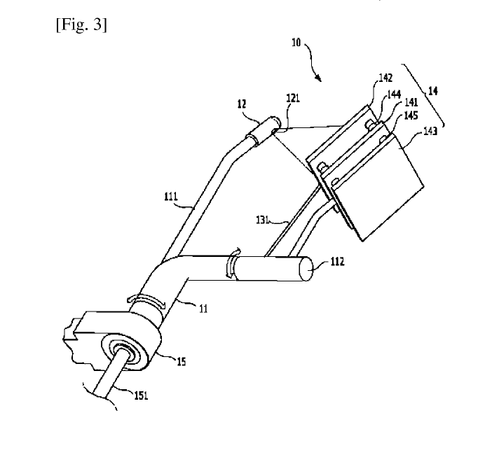

FIG. 3 is a diagram illustrating a case where an X-ray detection means of the intraoral X-ray detector assembly according to an embodiment of the present invention is brought close to the X-ray irradiation means which is inserted into the oral cavity.

Referring to FIG. 3, an aspect is illustrated in which the X-ray detection means 13 of the intraoral X-ray detector assembly 10 faces the X-ray emission port 121 of the X-ray irradiation means 12, while being brought close to the patient's dentition using the close-contact means 14.

Before or after the X-ray image pickup, the detection means support plate 142 of the contact means 14 is in close contact with the intermediate plate 141 side so as not to give discomfort to the patient, when the close-contact means 14 and the X-ray detection means 13 take the image pickup position. To this end, the elastic bodies 144 can be maximally compressed in a direction of being induced by the guides 145 as much as possible.

After the close-contact means 14 is located at a position facing the X-ray irradiation means 12, for preparation of the X-ray image pickup, the detection means support plate 142 of the contact means 14 moves toward the patient's dentition. This movement may be carried out while the elastic bodies 144 are pulled along the guides 145.

Although the detection means support plate 142 of the contact means 14 may come into contact with the patient's face, according to the embodiment, it can maintain a slight distance from the patient's face.

A portion of the detection means support plate 142 touching the patient's face can be shaped to match the patient's facial profile.

Although FIGS. 1 to 3 show that the X-ray detection means 13 is additionally attached to a side surface, on which the patient's face touches, of the two side surfaces of the detection means support plate 142, the X-ray detection means 13 may be integrally formed with the detection means support plate 142.

When the X-ray image pickup of one dentition is completed, the X-ray irradiation means 12 is capable of changing the X-ray emission direction by the rotation of the first shaft 111. For example, after the X-ray detection means 13 moves to the position away from the dentition of the patient along with the rotation of the second shaft 112, the close-contact means 14 changes the position toward the other dentition along with the rotation of the first shaft 111 and can move to a position facing the X-ray irradiation means 12 along with the rotation of the second shaft 112 again.

As described above, while the present invention has been described by the limited embodiments and drawings, the present invention is not limited to the above embodiments and various modifications and variations can be made by a person having ordinary knowledge in the art to which this invention pertains from these descriptions. Therefore, the idea of the present invention should be understood only by the claims set forth below, and all the equal or equivalent modifications would belong to the category of the spirit of the invention.

What is claimed is:

1. An intraoral X-ray detector assembly that provides X-ray detection signals to an X-ray imaging apparatus main body, the assembly comprising:
an X-ray irradiation means disposed to emit X-rays toward a dentition to be imaged within an oral cavity of a patient;
a close-contact means that can bring the X-ray detection means capable of detecting the X-rays close to the patient's face and can fix the X-ray detection means so as to face an X-ray emission direction of the X-ray irradiation means; and
a support means that supports a first shaft extending to the X-ray irradiation means and a second shaft extending to the close-contact means and is mechanically coupled to a connection means between the X-ray imaging apparatus main body.

2. The intraoral X-ray detector assembly of claim 1, wherein the close-contact means comprises: an intermediate plate coupled to the second shaft; a detection means support plate to which the X-ray detection means is coupled; and an elastic body interposed between the intermediate plate and the detection means support plate.

3. The intraoral X-ray detector assembly of claim 2, wherein the elastic body is a spring, and the close-contact means further comprises a guide that guides a compression or pulling direction of the spring between the intermediate plate and the detection means support plate.

4. The intraoral X-ray detector assembly of claim 1, wherein the support means is be mechanically coupled to the connection means so as to be attachable and detachable.

5. The intraoral X-ray detector assembly of claim 4, wherein the support means is mechanically coupled to the connection means so as to be attachable and detachable, when the X-ray irradiation means is in a direction positioned to face the patient's dentition.

6. The intraoral X-ray detector assembly of claim 1, wherein the support means is mechanically coupled to the connection means so as to be rotatable about a first rotary axis that coincides with an extension direction of the first shaft.

7. The intraoral X-ray detector assembly of claim 6, wherein the second shaft is mechanically coupled to the support means so as to be rotatable about the second rotary axis intersecting with the first rotary axis.

8. The intraoral X-ray detector assembly of claim 7, wherein the second shaft is mechanically coupled to the support means so as to be rotatable about the second rotary axis, between a position where the close-contact means faces the X-ray irradiation means and a movement position for changing an image pickup position of the close-contact means.

9. The intraoral X-ray detector assembly of claim 1, wherein the X-ray detection means is one of a one-dimensional X-ray line detector or a two-dimensional X-ray pixel array.
